(19) United States
(12) Patent Application Publication
(10) Pub. No.: US 2016/0270400 A1
(43) Pub. Date: Sep. 22, 2016

(54) Liposome-Attractant Formulations

(71) Applicant: LIPOTEC LABORATORIES LLC, Champaign, IL (US)

(72) Inventor: Kenneth L. Watkin, Champaign, IL (US)

(21) Appl. No.: 15/035,890

(22) PCT Filed: Nov. 11, 2014
(86) PCT No.: PCT/US14/65025

§ 371 Date: May 11, 2016

Related U.S. Application Data
(60) Provisional application No. 61/902,869, filed on Nov. 12, 2013.

(51) Int. Cl. A01N 47/24; A61K 31/7048; A01N 43/90; A61K 9/127; A61K 31/27

(52) U.S. Cl. CPC A01N 47/24; A61K 9/127; A61K 31/27; A01N 43/90; A61K 31/7048

(57) ABSTRACT

The invention provides liposomal-attractant formulations comprising pesticides or nematicides for control of pests. The formulations can be applied to pre- or post-emergent crops and to soil, plant media, plants, plant tissues and seeds. The liposomal-attractant formulations are also useful to treat or control pest or nematode infections of humans and animals.

This application claims the benefit of U.S. Ser. No. 61/902869, filed on Nov. 12, 2013, which is incorporated herein by reference in its entirety.

### BACKGROUND OF THE INVENTION

The use of nematicides and pesticides has been increasingly restricted over the past 30 years due to increased federal regulation and as concerns for human health and environmental safety has increased. The Food Quality Protection Action (1996) is resulting in further restrictions on the use of nematicides and pesticides. For example, the systemic nematicide fenamiphos was withdrawn from all uses in the United States in 2007. The use of aldicarb will be removed from markets by 2014. There is an urgent need to develop a low cost and quality enhancing technology, target oriented, environmentally compatible chemicals as well as suitable biological control methods for the control of pests, including insects and nematodes. There is also a need to develop, modify, or enhance existing technologies to control pests such as insects and nematodes.

### SUMMARY OF THE INVENTION

In one embodiment the invention provides liposome formulation comprising one or more pesticides or nematicides loaded in the aqueous core of liposomes, wherein one or more attractants for a pest or nematode are present within the lipid bilayer or bound to the surface of the liposome. The liposomes can be lyophilized or frozen. The one or more nematicides can be 2-methyl-2-(methylthio)propionaldehyde O-methylcarbamoyloxime, 2,3-Dihydro-2,2-dimethyl-7-benzofuranyl methylcarbamate, 2-methyl-2-(methylsulfonyl)propanal-O-(methylaminocarbonyl oximel, O,O-diethyl O-[p-(methylsulfinyl)phenyl]ester, Ethyl 4-methylth io-m-tolyl isopropylphosphoramidate, O-ethyl S,S-d ipropyl phosphorodithioate, Methyl N′N′-dimethyl-N-[(methylcarbamoyl)oxy]-1-thiooxamimidate, S-[[(1,1-dimethylethyl) thio] methyl]O,O-diethyl phosphorodithioate, thionazin, isazofos, ebufos, cleothocarb or combinations thereof. The lyophilized or frozen liposome can be loaded with about 1, 5, 10, 50, 100, 200, or 500 pg/ml of the one or more pesticides or nematicides. The liposome formulation can be dustable powder (DP), soluble powder (SP), water soluble granules (SG), water dispersible granules (WG), wettable powders (WP), granules (GR) (slow or fast release), soluble concentrates (SL), oil miscible liquids (OL), ultra-low volume liquids (UL), emulsifiable concentrates (EC), dispersible concentrates (DC), emulsions (both oil in water (EW) and water in oil (EO)), micro-emulsions (ME), suspension concentrates (SC), aerosols, fogging/smoke formulations, capsule suspensions (CS), powder for dry seed treatment (DS), a water soluble powder (SS), a water dispersible powder for slurry treatment (WS), a flowable concentrate (FS), a liquid solution (LS), a capsule suspension (CS), or combinations thereof. The liposomal formulation can further comprising a fertilizer. The pesticides or nematicides present in the aqueous core of a liposome can be administered at a same amount or concentration or a lower amount or concentration than the recommended administration amount or concentration of the pesticide or nematicide when administered in a non-liposomal formulation.

Another embodiment of the invention provides a method for reducing the number of pests or nematodes on or in plant media, soil, plants, plant tissues, or seeds. The method comprises administering to the plant media, soil, plants, plant tissues, or seeds an effective amount of a liposome formulation of the invention. The lyophilized liposomes can be rehydrated before they are administered to the plant media, soil, plants, plant tissues, or seeds. The lyophilized liposomes can be rehydrated with water, liquid fertilizer, or other suitable liquid. The methods can comprise administering about 5-fold, 10-fold, or 50-fold less pesticide or nematicide via the liposome formulation than is recommended for conventional, non-liposomal application of the same pesticides or nematicide. The plants or plants grown in the soil or plant media have increased root lengths, increased stalk diameter, increased stalk length, increased leaf number, increased leaf size, increased yield, or increased vigor as compared to plants or soil or plant media treated with non-liposomal formulations of the same one or more pesticides or nematicides of the administered liposome formulation. The liposome formulation can be administered in an amount from about 5 g/ha to about 2000 g/ha. The nematodes can be root-knot nematodes. The liposomal composition can be applied to seeds in an amount from about 0.001 g to about 10 kg per 100 kg of seeds.

Yet another embodiment of the invention provides a method of increasing root lengths, increasing stalk diameter, increasing stalk length, increasing leaf number, increasing leaf size of a plant, increasing yield, increasing plant vigor, or a combination thereof. The methods comprise administering a composition of the invention to the plant or to soil or plant media in which the plant is growing. Even another embodiment of the invention provides a method of increasing root lengths, increasing stalk diameter, increasing stalk length, increasing leaf number, increasing leaf size of a plant, increasing yield, increasing plant vigor or a combination thereof of a pesticide-treated or nematicide-treated plant or a plant grown in pesticide-treated or nematicide-treated soil or plant media. The method comprises administering one or more pesticides or nematicides to the plant or the soil or plant media, wherein the one or more pesticides or nematicides are present in an aqueous core of a liposome that has one or more attractants for a pest or nematode present within the lipid bilayer or bound to the surface of the liposome.

Still another embodiment of the invention provides a method of decreasing the amount of pesticide-induced or nematicide-induced damage to pesticide or nematicide treated plants or plants grown in pesticide-treated or nematicide-treated soil or plant media. The method comprises administering one or more pesticides or nematicides to the plant or the soil or plant media, wherein the one or more pesticides or nematicides are present in an aqueous core of a liposome that has one or more attractants for a pest or nematode present within the lipid bilayer or bound to the surface of the liposome. The pesticides or nematicides present in the aqueous core of a liposome can be administered at a same amount or concentration or a lower amount or concentration than the recommended administration amount or concentration of the pesticide or nematicide when administered in a non-liposomal formulation.

Another embodiment of the invention provides a method for reducing the number of nematodes on or in an animal, comprising administering to the animal an effective amount of a liposome formulation of the invention.

Yet another embodiment of the invention provides a method of making a liposomal formulation comprising one or more pesticides or nematicides loaded in the aqueous core of liposomes, wherein one or more attractants for a pest or nematode are present within the lipid bilayer of the liposome. The method comprises:
(a) using an extruder to make liposomes with one or more one or more pesticides or nematicides loaded in the aqueous core of liposomes; and either:
   (i) adding the liposomes to a concentrator with one or more lipophilic pesticide or nematode attractants and increasing the pressure within the concentrator such that the one or more attractants are located within the lipid bilayers of the liposomes; or
   (ii) linking one or more pesticide or nematode attractants to the surface of the liposomes.

### BRIEF DESCRIPTION OF THE DRAWINGS

FIG. 1 shows nematicidal activity of 100 μg oxamyl-liposome formulation on root-knot nematodes (% living after treatment).

FIG. 2 shows the effect of pre-emergent application of liposomal formulations of Avid 0.15 on tomato stalk height.

FIG. 3 shows the effect of 5 μg and 1 μg liposomal abamectin formulations (“Aba-lipo”) 5 μg and 1 μg non-liposomal abamectin formulations (“Aba only”) on gall formation.

FIG. 4 shows the effect of 5μg and 1 μg liposomal abamectin formulations (“Aba-lipo”) 5 μg and 1 μg non-liposomal abamectin formulations (“Aba only”) on root necrosis.

FIG. 5 shows the effect of 5μg and 1 μg liposomal abamectin formulations (“Aba-lipo”) 5 μg and 1 μg non-liposomal abamectin formulations (“Aba only”) on root length.

FIG. 6 shows the results of different dose levels of abamectin or liposomal encapsulated abamectin on the number of galls, percentage of root necrosis, and root length in inches.

FIG. 7A-C shows graphs of the number of galls, percentage of root necrosis, and root length (in inches) in tomato plants treated with abamectin or liposomal encapsulated abamectin.

FIG. 8 shows the statistical comparisons between the treatment types of tomato plants treated with abamectin or liposomal encapsulated abamectin.

FIG. 9 shows the probabilistic modeling for 1 acre of soybeans when using abamectin encapsulated liposomes.

### DETAILED DESCRIPTION OF THE INVENTION

As used herein, the singular forms “a,” “an”, and “the” include plural referents unless the context clearly dictates otherwise.

Liposomes have received widespread attention as a carrier system for therapeutically active compounds, due to their unique characteristics such as capability to incorporate hydrophilic and hydrophobic drugs, good biocompatibility, low toxicity, lack of immune system activation, and targeted delivery of bioactive compounds to the site of action (Voinea et al., J. Cell Mol. Med. 6:465 (2002)). Additionally, some achievements since the discovery of liposomes are controlled size from microscale to nanoscale and surface-engineered polymer conjugates functionalized with peptide, protein, and antibody. Progress in liposome drug delivery has led to the commercialization of liposomal anticancer drug formulations (e.g., Doxil, DaunoXome).

Liposomal formulations have now been developed that are suitable for protecting plants and plant organs (including fruits and seeds), for increasing the harvest yields, for improving the quality of the harvested material, for controlling animal pests, in particular insects, arachnids, helminths, nematodes and molluscs, which are encountered in agriculture, in horticulture, in animal husbandry, in forests, in gardens and leisure facilities, in the protection of stored products and of materials, and in the hygiene sector. The liposome formulations can be frozen or lyophilized to produce a long lasting and storable composition which can then be further processed to meet the needs of a given application.

### Advantages of Compositions of the Invention

Administration of the liposomal compositions of the invention can provide one or more advantageous properties to soil, plant medium, seeds, plants or plant tissues. Examples of such advantageous properties include a broadening of the spectrum of pesticidal activity to other pests; a reduction in the rate of application of the active ingredients; adequate control of the pests with the aid of combinations of active ingredients, even at a rate of application at which the individual active ingredients are totally ineffective; advantageous behavior during formulating and/or upon application, for example upon grinding, sieving, emulsifying, dissolving or dispersing; increased storage stability; improved stability to light; increased advantageous degradability; improved toxicological and/or ecotoxicological behavior; improved crop characteristics including: emergence, crop yields, more developed root system (including longer roots), tillering increase, increase in plant height, increase in stalk circumference, bigger leafs, more leaves, less dead basal leaves, stronger tillers, greener leaf color, less fertilizers needed, less seeds needed, more productive tillers, earlier flowering, early grain, seed or fruit maturity, less plant verse (lodging), increased shoot growth, improved plant vigor, and early germination; or any other advantages familiar to a person skilled in the art.

An improvement in the growing (or growth) characteristics of a plant can be measured in many ways, but ultimately results in a better production of the plant, for example, an improved yield, improved vigor of the plant or quality of the harvested product from the plant. An improved yield of a plant relates to an increase in the yield of a product (e.g., as measured by plant biomass, grain, seed or fruit yield, protein content, carbohydrate or oil content or leaf area) of the plant by a measurable amount over the yield of the same product of the plant produced under the same conditions, but without the application of compositions of the invention or compared with application of conventional non-liposomal pesticides or nematicides. Yield can be increased by at least about 0.5, 1, 2, 3, 4, 5, 10, 15% or more. Yield can be expressed in terms of an amount by weight or volume of the plant or a product of the plant on some basis. The basis can be expressed in terms of time, growing area, weight of plants produced, or amount of a raw material used.

An improved vigor of a plant is an increase or improvement of the vigor rating, the stand (the number of plants per unit of area), plant height, stalk circumference, plant canopy, visual appearance (such as greener leaf color), root rating, emergence, protein content, increased tillering, bigger leafs, more leaves, less dead basal leaves, stronger tillers, less fertilizer needed, less seeds needed, more productive tillers, earlier flowering, early grain or seed maturity, less plant verse (lodging), increased shoot growth, earlier germination, or any combination of these factors, by a measurable or noticeable amount over the same factor of the plant produced under the same conditions, but without the administration of the instant compositions or with application of conventional non-liposomal pesticides.

### Pests

The compositions of the invention can be used to prevent infection by or reduce the numbers of plant pests in or on soil or other plant medium and to prevent infection or reduce the numbers of plant pests on plants or plant material such as roots, fruits and seeds. In another embodiment of the invention, the compositions of the invention reduce the damaging effect of plant pests on the plant by, for example, killing, injuring or slowing the activity of the pest. Plant pests include, for example, insects, arachnids, helminths, nematodes, molluscs, bacteria, fungi, mites, oomycytes and protozoa. Compositions of the invention can be used to control, kill, injure, paralyze, or reduce the activity of one or more of any of these pests in their egg, larvae, adult, juvenile, or desiccated forms.

Nematodes that damage plants include, for example, Meloidogyne spp. (root-knot), Heterodera spp., Globodera spp., Pratylenchus spp., Helicotylenchus spp., Radopholus similis, Ditylenchus dipsaci, Rotylenchulus reniformis, Xiphinema spp., Aphelenchoides spp. and Belonolaimus longicaudatus.

Plant parasitic nematodes are small, aquatic, microscopic roundworms that live in films of water surrounding soil particles and plant roots. The presence of a water film is essential to the nematode for locomotion and maintenance of body fluids. The body of the nematode, when inflated with fluids, acts like a skeleton, preventing internal collapse. In dry soils body fluids are lost, the body wall collapses, and many nematodes die as a result of dehydration. However, some can survive desiccation in a suspended state for long periods, and come back to life when soil water conditions are restored. In the dried state, nematodes are more resistant to high soil temperature and nematicides. Nematodes feed on the roots or foliar tissues of plants. In many parts of the world nematodes are a major limiting factor for agricultural production, causing serious reduction in crop quantity, quality, or harvest uniformity. All fruit and vegetable crops are susceptible to nematodes. Total crop failures frequently occur when crops are planted into areas with high nematode population levels. Plant symptoms that develop in response to nematode parasitism are generally those associated with root dysfunction. Development of small, stunted, and chlorotic plants generally reflects reduced water and nutrient uptake caused by injury to the root system. Correspondingly, root damage generally increases with nematode infestation level, particularly where plants are grown on fine to coarse textured, sandy soils with low water holding capacity. Plant-parasitic nematodes cause yield suppression in many crops species. Estimates of nematode damage to specific crops ranged from 3.3% to 20.6%, with a mean of 12.3%. Annual production losses at the farm gate were $121 billion globally and $9.1 billion in the United States (Sasser, J. N. & Freckman, 1987 In: Veech & Dickson, eds. Vistas on Nematology p. 7-14, Hyattsville Md., US, Society of Nematologists).

The root-knot group Meloidogyne spp of nematodes are particularly important to control (Sasser, Plant Disease, 104:36 (1980)). Their worldwide distribution, extensive host ranges and involvement with fungi, bacteria, and viruses in disease complexes rank them among the top major plant pathogens affecting the global food supply. Collectively, the various species of root-knot attack nearly every crop grown. The most common species are M. incognita, M. arenaria, M. hapla and M. javanica (Sasser, Phytopathology, 42:216 (1952); Sasser, Bull. Md. Agric. Exp. Stn. A-77 (Techn) p. 31 (1954)). Not only are yields greatly affected, but production quality is also reduced. Infections by root-knot nematode cause decline in the host, and under some conditions, may kill the plant (Sasser, 1980). Infected plants may be stunted and chlorotic, may usually wilt easily, and become unproductive. However, the extent of damage caused by root-knot nematode infections vanes with the host, the timing of infection, and the cultural conditions present. Root-knot nematode infection is easy to identify because of the swellings in roots that look like “knots.” The swellings become large and easy to see on some hosts such as squash and tomato, but may be smaller and less conspicuous on others such as the ‘Chile’ pepper. Multiple infections on one root result in a swollen, rough appearance. Root-knot nematodes are very small and can only be observed using a microscope.

Unlike free-living nematodes that are numerous in all soils, plant parasitic nematodes must feed on a plant host in order to complete their life cycle. Root-knot nematodes are soil borne and feed on roots (Taylor & Sasser, 1978, Biology, Identification, and Control of Root-Knot Nematodes (Meloidogyne species) Raleigh, N.C., USA, NC State University Graphics, 111 pp.). Their life cycle includes egg, juvenile and adult stages. Eggs hatch into juveniles that infect plant roots and take nutrients from the plant as they mature, causing the characteristic knots or swellings to form. Root-knot nematodes feed by means of a stylet, a retractable mouthpart used for piercing and feeding. Those that enter the root and develop into females are sedentary, become much enlarged, and lay hundreds of eggs in a sac on the root surface. In moist soils above 80° F., root-knot nematodes can go from egg to adult in about 25 days. In adverse conditions, the eggs can persist in the soil for long periods of time ranging from months to years.

Nematodes are most active in warm weather in moist, but well aerated, sandy soils in the presence of host plants. They are most abundant in the upper foot of soils, but will follow roots several feet deep. Three options exist for the management of root-knot nematodes: crop rotation, host plant resistance, and nematicides. For example, rotating corn with a non-host crop such as alfalfa or oats may be effective in reducing root-knot nematode populations. Because different species have different host ranges, it is always good practice to identify the particular species in the field before deciding on crop rotation as a management strategy.

Plants that are non-hosts of M. incognita can serve as good hosts for M. arenaria or M. javanica. Fields with successive seasons of corn will suppress populations of northern root-knot nematode, M. hapla, but at the same time this scheme may enhance populations of other rootknot, stubby-root, lesion, sting, lance, and ring nematodes. Resistant corn cultivars are currently unavailable for southern root-knot nematode, M. incognita; however, there are a few commercial cultivars that are resistant to M. arenaria and M. javanica. Lack of good cultural management alternatives leaves nematicides as the primary nematode management tool for most corn, soybean, vegetable, cotton, and fruit tree growers.

Among the crops with the greatest estimated losses due to nematode parasitism are corn, cotton, cucurbits, leguminous vegetables, peanut, solanaceous vegetables, soybean, sugarcane, and tobacco.

Insects cause two types of damage to plants. The first type of damage is direct injury done to the plant by the insect, which eats leaves or burrows into plant tissues. There are a multitude of insect species of this type, both larvae and adults, among orthopterans, homopterans, heteropterans, coleopterans, lepidopterans, and dipterans. The second type of damage is indirect damage where the insect itself does little or no harm but transmits a bacterial, viral, or fungal infection to a plant. Insects that cause these two types of damage to plants include, for example, Coleoptera (beetles, weevils), Cerambycidae (long-horned beetles), Chrysomelidae (leaf beetles), Coccinellidae (lady beetles), Curculionidae (snout beetles, weevils, billbugs), Elateridae (click beetles), Meloidae (blister beetles), Scarabaeidae (scarab beetles), Tenebrionidae (darkling beetles), Diptera (flies), Anthomyiidae (root maggot flies), Cecidomyiidae (midges), Hemiptera suborder heteroptera (true bugs), Lygaeidae (seed bugs, chinch bugs), Miridae (plant bugs, lygus bugs), Pentatomidae (stink bugs), Hemiptera suborder homoptera (aphids, whiteflies, leafhoppers, scales), Aleyrodidae (whiteflies), Aphididae (aphids), Cercopidae (spittlebugs), Cicadellidae (leafhoppers), Membracidae (treehoppers), Lepidoptera (moths, butterflies), Noctuidae (cutworm moths), Pyralidae (snout and grass moths), Sphingidae (sphinx moths), Orthoptera (grasshoppers and crickets), Acrididae (short-horned grasshoppers), Gryllidae (crickets), Gryllotalpidae (mole crickets), Thysanoptera (thrips), Thripidae (common thrips), Acarina (mites), Tetranychidae (spider mites).

Arachnids such as earth mites (Penthaleidae), thread-footed mites (Tarsonemidae) and gall and rust mites (Eriophyoidea) can also cause damage to plants.

Molluscs, including those in the gastropod class and those in the subclass pulmonata, can cause damage to plants. Molluscs also include, for example, snails and slugs, such as Ampullariidae spp.; Arion spp. (A. ater, A. circumscriptus, A. hortensis, A. rufus); Bradybaenidae spp. (Bradybaena fruticum); Cepaea spp. (C. hortensis, C. nemoralis); Ochlodina; Deroceras spp. (D. agrestis, D. empiricorum, D. laeve, D. reticulatum); Discus spp. (D. rotundatus); Euomphalia spp.; Galba spp. (G. trunculata); Helicelia spp. (H. itala, H. obvia); Helicidae spp. (Helicigona arbustorum); Helicodiscus spp.; Helix spp. (H. aperta); Limax spp. (L. cinereoniger, L. flavus, L. marginatus, L. maximus, L. tenellus); Lymnaea spp.; Milax spp. (M. gagates, M. marginatus, M. sowerbyi); Opeas spp.; Pomacea spp. (P. canaticulata); ValIonia spp. and Zanitoides.

Any type of plant, plant tissue, seed or plant media, or soil can be treated with the compositions of the invention. Plants include algae, bryophytes, tracheophytes, and angiosperms. Angiosperms include, for example, flowering plants, cycads, Ginkgo biloba, and conifers. Plants include seedlings, mature plants, trees and turf. Plant tissues can include, for example, roots, leaves, stems, flowers, seeds, and fruits.

### Nematicides and Pesticides

Pesticides are active agents that kill or inhibit the growth of pests such as insects, arachnids, helminths, nematodes, molluscs, bacteria, fungi, mites, oomycytes and protozoa. Liposomes of the invention can comprise pesticides, including nematicides, and herbicides. Examples of pesticides and herbicides that can be used in the liposomal formulations of the invention include, for example, 1-bromo-3-chloro-5,5-dimethylhydantoin, 2,4-D Amine, 2,4-D low volatile ester, 2,4-DB, 2,4-D+fenoxaprop-p-ethyl+MCPA+thifensulfuron methyl, abamectin, acephate, acetamiprid, acetic acid, Agrobacterium radiobacter, aluminum phosphide, amitraz, amitrole, ancymidol, anilazine, atrazine, atrazine & bentazon, atrazine & etolachlor, azinphos-methyl, azoxystrobin, Bacillus thuringiensis (Bt), bendiocarb, bensulide, bentazon, boscalid, brodifacoum, bromadiolone, bromethalin, bromoxynil, bromoxynil+MCPA, bromoxynil+2,4-D ester, captan, captan+diazinon+thiophanate-methyl, captan+thiophanate methyl, carbaryl, carbathiin, carbathiin+captan, carbathiin+clothianidin+thiram+metalaxyl, carbathiin+imidacloprid+thiram, carbathiin+oxycarboxin+thiram, carbathiin+thiabendazole, carbathiin+thiram, carbofuran, chloroneb, chlorophacinone, chlorothalonil, chlorothalonil+propamocarb HCl, chlorpropham, chlorpyrifos, chlormequat chloride, clethodim, clodinafop-propargyl, clodinafop-propargyl+thifensulfuron-methyl+tribenuron-methyl, clofentezine, clopyralid, clopyralid+glyphosate, clopyralid+MCPA ester, clothianidin, clothianidin+carbathiin+thiram+metalaxyl, copper 8-quinolinolate, copper hydroxide, copper oxychloride, copper sulphate, cyfluthrin, cyhalothrin-lambda, cymoxanil, cymoxanil+famoxadone, cypermethrin, cyprodinil, cyromazine, daminozide, dazomet, deltamethrin, desmedipham+phenmedipham, diazinon, diazinon+captan, diazinon+captan+thiophanate-methyl, diazinon+cypermethrin, dicamba, dicamba+atrazine, dicamba+glyphosate, dicamba+MCPA, dicamba+mecoprop+2, 4-D dicamba+mecoprop, dicamba+mecoprop+MCPA, dicamba+2, 4-D, dichlobenil, diclofop-methyl, diclofop-methyl+bromoxynil, dicloran, dichloropropene, dichloropropene+chlorpicrin, dichlorprop+2,4-D dichlorvos, dichlorvos+pyrethrins+piperonyl butoxide, dichlorvos+pyrethrins+piperonyl butoxide+di-n-propylisocinchomeronate Dicofol, didecyl dimethyl ammonium chloride, didecyl dimethyl ammonium chloride+dimethyl benzyl ammonium chloride, difenoconazole, difenocanazole+metalaxyl-M, difenoconazole+metalaxyl-M+fludioxonil, difenoconazole+thiamethoxam+metalaxyl-M+fludioxonil, difenzoquat, diflubenzuron, dimethoate, dimethomorph, dimethomorph+mancozeb, dinocap+mancozeb, diphacinone, diquat, diuron, dodemorph-acetate, dodine, endosulfan, EPTC, ethalfluralin, ethametsulfuron-methyl, ethephon, etridiazole, famoxadone+cymoxanil, fatty acids, fenbuconazole, fenbutatin-oxide, fenhexamid, fenoxaprop-p-ethyl, fenoxaprop-p-ethyl+bromoxynil+MCPA, fenoxaprop-p-ethyl+MCPA+thifensulfuron methyl, fenoxaprop-p-ethyl+MCPA+2,4-D+thifensulfuron methyl, fenaoxprop-p-ethyl+thfensulfuron methyl+tribenuron methyl, ferbam, florasulam+glyphosate, florasulam+MCPA ester, fluazifop-p-butyl, fludioxonil+difenoconazole+metalaxyl-M, fludioxonil+difenoconazole+thiamethoxam+metalaxyl-M, fluroxypyr, fluroxypyr+2,4-D ester, fluroxypyr+MCPA ester, fluroxypyr+clopyralid+MCPA ester, flusilazole, folpet, formaldehyde, formetanate hydrochloride, fosetyl-aluminum, gibberellic acid, gibberellins+benazladenine, glufosinate ammonium, glyphosate, glyphosate+2,4-D glyphosate+dicamba, glyphosate+florasulam, Heterorhabditis megidis, hexazinone, imazamethabenz, imazamox+imazethapyr, imazethapyr, imazethapyr+pendimenthalin, imidacloprid, imidacloprid+carbathiin+thiram, iprodione, isoxaben, kinoprene, kresoxim-methyl, lime sulphur, linuron, malathion, maleic hydrazide, mancozeb, mancozeb+dimethomorph, mancozeb+dinocap, mancozeb+metalaxyl-M, mancozeb+zoxamide, maneb, MCPA+MCPB, MCPA dimethylamine, MCPA dimethyl amine+dicamba+mecoprop, MCPA ester, MCPA ester+bromoxynil, MCPA ester+clopyralid, MCPA ester+fenoxaprop-p-ethyl+thifensulfuron methyl, MCPA ester+fenoxaprop-p-ethyl+2,4-D+thifensulfuron methyl, MCPA ester+fenoxaprop-p-ethyl+bromoxynil, MCPA ester+florasulam, MCPA ester+fluroxypyr, MCPA potassium salt, MCPA potassium salt+dicamba, MCPA sodium salt, MCPB, MCPB+MCPA, mecoprop, mecoprop+MCPA dimethyl amine+dicamba, mefenoxam (s-isomer)+etalaxy-M, metalaxyl, metalaxyl-M+chlorothalonil, metalaxyl-M+difenoconazole, metalaxyl-M+mancozeb, metaldehyde, metam sodium, methamidophos, methomyl, methomyl+Z-9 tricosene, methoxyfenozide, methoprene, methyl bromide, methyl bromide & chloropicrin, metiram, metolachlor/s-metolachlor, metolachlor+atrazine, metribuzin, metribuzin+tribenuron methyl, metsulfuron methyl, mineral & vegetable oil, myclobutanil, NAA, naled, napropamide, naptalam, napthalene acetamide, nicosulfuron, nicotine, oxadiazon, oxamyl, oxine benzoate, oxycarboxin, oxycarboxin+carbathiin+thiram, oxyfluorfen, paclobutrazol, paraquat, pendimethalin, pendimenthalin+imazethapyr, permethrin, permethrin+pryethrins+piperonyl butoxide, piperonyl butoxide+dichlorvos+pyrethrins, phenmediphan+desmedipham, phosalone, phosmet, pirimicarb, prohexadione ca, prometryne, propamocarb hydrochloride, propamocarb HCl+chlorothalonil, propanil, propiconazole, propiconazole+azoxystrobin, propyzamide, putrescent whole egg solids, pyraclostrobin, pyrethrins, pyrethrins+piperonyl butoxide, pyrethrins+piperonyl butoxide+dichlorvos, pyrethrins+piperonyl butoxide+malathion, pyridaben, quinclorac, quinclorac+thifensulfuron methyl+tribenuron methyl, quintozene (PCNB), rimsulfuron, sethoxydim, simazine, soaps, spinosad, Steinernema feltiae, stoddard solvent, streptomycin sulfate, strychnine, sulphur, tebuconazole+thiram, tebufenozide, tefluthrin, terbacil, terbufos, tetrachlorvinphos, thiabendazole, thiabendazole+carbathiin, thiamethoxam+difenoconazole+metalaxyl-M+fludioxonil, thifensulfuron methyl, thifensulfuron methyl+tribenuron methyl, thifensulfuron methyl+tribenuron methyl+quinclorac, thifensulfuron methyl+MCPA ester+fenoxaprop-p-ethyl, thifensulfuron methyl+tribenuron methyl+fenaoxprop-p-ethyl, thifensulfuron-methyl+tribenuron-methyl+clodinafop-propargyl, thiophanate methyl, thiophanate methyl+captan, thiophanate-methyl+diazinon+captan, thiophanate methyl+imidacloprid+mancozeb, thiram, thiram+carbathiin, thiram+carbathiin+oxycarboxin, thiram+carbathiin+imidacloprid, thiram+carbathiin+clothianidin+metalaxyl, thiram+tebuconazole, thiram+triticonazole, tralkoxydim, tralkoxydim+bromoxynil+MCPA, tralkoxydim+clopyralid+MCPA, triadimenol, triallate, triallate+trifluralin, tribasic copper sulphate, tribenuron methyl, tribenuron methyl+2,4-D, tribenuron methyl+metribuzin, tribenuron-methyl+thifensulfuron-methyl+clodinafop-propargyl, tribenuron methyl+thifensulfuron methyl, tribenuron methyl+thfensulfuron methyl+fenaoxprop-p-ethyl, tribenuron methyl+thifensulfuron methyl+quinclorac, trichlorfon, trifluralin, trifluralin+triallate, triforine, trinexapac-ethyl, triticonazole+thiram, uniconazole, vinclozolin, warfarin, warfarin+sulfaquinoxaline, zinc phosphide, zineb, ziram, zoxamide+mancozeb or use of each of these individually or combinations thereof.

Nematicides are, by definition, chemicals that kill nematodes (−cides). Two broad categories of nematicides are currently registered and available for use (Whitehead, 1998, Plant Nematode Control. CAB International, Walling Ford, UK). The classification system is based upon the way these chemicals move in soil. Fumigant nematicides, including methyl bromide, methyl iodide, chloropicrin, ethylene dibromide, 1,3-dichloropene, dimethyl dibromide and metam sodium and potassium, dazomet, methyl isothiocyanate, are formulated as liquids which rapidly vaporize and move through open air spaces in soil as a gas. Non-fumigant nematicides, including 2-methyl-2-(methylthio)propionaldehyde O-methylcarbamoyloxime (Temik®, Bayer CropScience), 2,3-Dihydro-2,2-dimethyl-7-benzofuranyl methylcarbamate (Furadan), 2-methyl-2 (methylsulfonyl)propanal-O-(methylaminocarbonyl oximel (Standak™, BASF), O,O-diethyl 0-[p-(methylsulfinyl)phenyl]ester (Dasanit), Ethyl 4-methylthio-m-tolyl isopropylphosphoramidate (Nemacur, Makhteshim Agan Group), O-ethyl S,S-dipropyl phosphorodithioate (MOCAP®, Bayer CropScience), Methyl N′N′-dimethyl-N-[(methylcarbamoyl)oxy]-1-thiooxamimidate (Vydate®, Dupont), and S-[[(1,1-dimethylethyl) thio] methyl]O,O-diethyl phosphorodithioate (Counter), thionazin (Nemafos), Isazofos (Miral), Ebufos (Rugby), Cleothocarb (Lance) are organophosphates and/or carbamates. The non-fumigant nematicides are often further classified as contact or systemic nematicides, depending on whether they kill nematodes in soil by contact, or are taken up by the plant first and affect nematodes when they feed from cellular fluids within the plant.

Any pesticide or nematicide can be loaded into a liposome of the invention.

### Liposomes

Liposomes of the invention include, for example, small unilamellar vesicles

(SUVs) formed by a single lipid bilayer, large unilamellar vesicles (LUVs), which are vesicles with relatively large particles formed by a single lipid bilayer, and multi-lamellar vesicles (MLVs), which are formed by multiple membrane layers. Liposomes and nanoliposomes (submicron bilayer lipid vesicles) can be of any particle size, for example the mean particle diameter can be about 10 to about 2000 nm. In one embodiment of the invention, the mean particle diameter is about 10, 20, 25, 30, 40, 50, 100, 200, 300, 400, 500, 600, 700, 800, 900, 1,000, 1,250, 1,500, 1,750, 2,000 nm (or any range between about 10 and about 2,000 nm) or more. In one embodiment of the invention, the mean particle diameter is about 2,000, 1,750, 1,500, 1,250, 1,000, 900, 800, 700, 600, 500, 400, 300, 200, 100, 50, 40, 30, 25, 20, 10 nm (or any range between about 2,000 and 10 nm) or less. The mean particle diameter may be about 20 to about 1,000 nm, about 100 to about 1,500 nm, about 100 to about 1,000 nm, about 100 to about 700 nm, about 200 to about 2,000 nm, about 1,000 to about 2,000 nm, or about 750 to about 1,500 nm. Particle diameter refers to the diameter of a particle measured by dynamic light scattering.

Liposome manufacture comprises, for example, drying down of the lipids from organic solvents, dispersion of the lipids in aqueous media, purification of the resultant liposomes, and analysis of the final product. Some methods of liposome manufacture include, for example, extrusion methods, the Mozafari method, the polyol dilution method, the bubble method, and the heating method. Pesticides can be entrapped in lipid vesicles by any method including, for example, reverse-phase evaporation technique, ether injection/vaporization technique and the freeze-thaw method.

Examples of lipids that can be used to make liposomes of the invention include soybean lecithin, hydrogenated soybean lecithin, egg yolk lecithin, phosphatidylcholines, phosphatidylserines phosphatidylethanolamines, phosphatidyl inositols, sphingomyelins, phosphatidic acids, long-chain alkyl phosphates, gangliosides, glycolipids, phosphatidyl glycerols, and cholesterols. Phosphatidylcholines include, for example, dimyristoylphosphatidylcholine, dipalmitoylphosphatidylchol ine, and distearoyl phosphatidylcholine. Phosphatidylserines include, for example, dipalmitoyl phosphatidylserine, dipalmitoyl phosphatidylserine (sodium salt), and phosphatidylserine (sodium salt) derived from bovine brain. Phosphatidylethanolamines include, for example, dimyristoyl phosphatidylethanolamine, dipalmitoyl phosphatidylethanolamine, and distearoyl phosphatidylethanolamine. Phosphatidyl inositols include, for example, phosphatidylinositol (sodium salt) derived from wheat. Sphingomyelins include for example, sphingomyelin derived from bovine brain. Phosphatidic acids and long-chain alkyl phosphates include, for example, dimyristoyl phosphatidic acid, dipalmitoyl phosphatidic acid, distearoyl phosphatidic acid, and dicetyl phosphate. Gangliosides include, for example, ganglioside GM1, ganglioside GD1a, and ganglioside GT1b. Glycolipids include, for example, galactosyl ceramide, glucosyl ceramide, lactosyl ceramide, phosphatide, and globoside. Phosphatidyl glycerols include, for example, dimyristoyl phosphatidylglycerol, dipalmitoyl phosphatidylglycerol, and distearoyl phosphatidylglycerol. One or more types of lipids can be used to make a liposome of the invention.

A liposome composition of the invention can comprise about 0.001, 0.01, 0.1, 1.0, 2, 3, 4, 5, 6, 7, 8, 9, 10, 15, 20, (or any range between about 0.001 and 20) or more wt % of a pesticide, for example a nematicide. A liposome composition of the invention can comprise about 20, 15, 10, 9, 8, 7, 6, 5, 4, 3, 2, 1.0, 0.1, 0.01, 0.001 (or any range between about 20 and 0.001) or less wt % of a pesticide, for example a nematicide. For example, a liposome composition can comprise about 0.001 to about 0.01 wt %, about 0.01 to about 0.1 wt %, about 0.1 to about 1 wt %, about 1 to about 5 wt %, or about 5 to about 10 wt %, about 10 to about 20 wt % of a pesticide or nematicide. A liposome composition can comprise about 2, 3, 4, 5, 7, 10, 12, 15, 16, 17, 18 wt % (or any range between about 2 and 18 wt %) or more lipid phase and about 82, 83, 84, 85, 88, 90, 93, 95, 96, 97, 98 wt % (or any range between about 82 and 98 wt %) aqueous phase. The lipid phase may comprise about 2, 5, 10, 15, 20, 30, 40, 50, 60, 70, 75, or 80 wt % phospholipids, for example about 25 to about 44 wt % phospholipids.

A liposome of the invention can be loaded with about 1, 5, 10, 50, 100, 200, or 500, 1,000, 2,000 (or any range between about 1 and 2,000) or more pg/ml of nematicides or pesticides. A liposome of the invention can be loaded with about 2,000, 1,000, 500, 200, 100, 50, 10, 5, 1 (or any range between about 2,000 and 1) or less pg/ml of nematicides or pesticides.

The lipid phase may optionally comprise one or more additional agents such as thickeners, gelling agents, preservatives, stabilizers, wetting agents, pH buffering agents, emulsifiers, stearylamine, phosphatidic acid, dicetyl phosphate, sterols, cholesterol, cholesterol stearate, lanolin extracts, hydroxypropylmethycellulose, carboxymethycellulose, sodium acetate, sorbitan monolaurate, triethanolamine oleate, and sorbitol. An additional agent may be present at about 0.01, 0.1, 1, 2, 5, 7, 10, 12, or 15 wt % of the lipid phase.

A liposome composition can also include one or more additives to improve the biological performance of the composition (for example by improving wetting, retention or distribution on surfaces; resistance to rain on treated surfaces; or uptake or mobility of a liposome formulation). Such additives include surface active agents, spray additives based on oils, for example certain mineral oils or natural plant oils (such as soy bean and rape seed oil), and blends of these with other bio-enhancing adjuvants (ingredients which may aid or modify the action of a liposome formulation).

After formation and loading of liposomes with one or more pesticides including, for example one or more nematicides, the liposomes can be freeze-dried or lyophilized. See U.S. Pat. No. 4,311,712. The liposomes can be reconstituted on contact with water or another liquid. Other components can be added to the lyophilized or reconstituted liposomes, for example, water, fertilizer, pesticides, or herbicides.

In one embodiment of the invention the pest, for example, a nematode, ingests the liposomes of the invention. In another embodiment of the invention, the liposome delivers the pesticide within the liposome to soil, plant media, plant, plant tissue, seed or fruit via slow release from the liposome, where the pest or nematode then comes in contact with the pesticide.

Due to their structure, chemical composition and colloidal size, all of which can be well controlled during preparation protocols, liposomes exhibit several properties that can be useful in various applications. The most important properties are colloidal and special membrane and surface characteristics. The colloidal stable liposomes make them work well as a carrier of different molecules, i.e., drug molecules. They also include bilayer phase behavior, its mechanical properties and permeability, charge density, presence of surface bound or grafted polymers, or attachment of special ligands, respectively. Additionally, due to their amphiphilic character, liposomes are a powerful solubilizing system for a wide range of compounds. Liposomes have a non-equilibrium structure and are less sensitive to external changes than equilibrium structures, such as micelles. In addition to these physico-chemical properties, liposomes exhibit many special biological characteristics, including (specific) interactions with biological membranes and various cells.

The liposome formulations can be chosen from a number of formulation types, including dustable powders (DP), soluble powders (SP), water soluble granules (SG), water dispersible granules (WG), wettable powders (WP), granules (GR) (slow or fast release), soluble concentrates (SL), oil miscible liquids (OL), ultra-low volume liquids (UL), emulsifiable concentrates (EC), dispersible concentrates (DC), emulsions (both oil in water (EW) and water in oil (EO)), micro-emulsions (ME), suspension concentrates (SC), aerosols, fogging/smoke formulations, capsule suspensions (CS) and seed treatment formulations. The formulation type chosen in any instance will depend upon the particular purpose envisaged and the physical, chemical and biological properties of the liposome formulation.

Dustable powders (DP) may be prepared by mixing a liposome formulation with one or more solid diluents (for example natural clays, kaolin, pyrophyllite, bentonite, alumina, montmorillonite, kieselguhr, chalk, diatomaceous earths, calcium phosphates, calcium and magnesium carbonates, sulfur, lime, flours, talc and other organic and inorganic solid carriers) and mechanically grinding the mixture to a fine powder.

Soluble powders (SP) may be prepared by mixing a liposome formulation with one or more water-soluble inorganic salts (such as sodium bicarbonate, sodium carbonate or magnesium sulfate) or one or more water-soluble organic solids (such as a polysaccharide) and, optionally, one or more wetting agents, one or more dispersing agents or a mixture of said agents to improve water dispersibility/solubility. The mixture is then ground to a fine powder. Similar compositions may also be granulated to form water soluble granules (SG).

Wettable powders (WP) may be prepared by mixing a liposome formulation with one or more solid diluents or carriers, one or more wetting agents and, preferably, one or more dispersing agents and, optionally, one or more suspending agents to facilitate the dispersion in liquids. The mixture is then ground to a fine powder. Similar compositions may also be granulated to form water dispersible granules (WG).

Granules (GR) may be formed either by granulating a mixture of a liposome formulation and one or more powdered solid diluents or carriers, or from pre-formed blank granules by absorbing a liposome formulation (or a solution thereof, in a suitable agent) in a porous granular material (such as pumice, attapulgite clays, fuller's earth, kieselguhr, diatomaceous earths or ground corn cobs) or by adsorbing a liposome formulation (or a solution thereof, in a suitable agent) on to a hard core material (such as sands, silicates, mineral carbonates, sulfates or phosphates) and drying if necessary. Agents which are commonly used to aid absorption or adsorption include solvents (such as aliphatic and aromatic petroleum solvents, alcohols, ethers, ketones and esters) and sticking agents (such as polyvinyl acetates, polyvinyl alcohols, dextrins, sugars and vegetable oils). One or more other additives may also be included in granules (for example an emulsifying agent, wetting agent or dispersing agent).

Dispersible Concentrates (DC) may be prepared by dissolving a liposome formulation in water or an organic solvent, such as a ketone, alcohol or glycol ether. These solutions may contain a surface active agent (for example to improve water dilution or prevent crystallization in a spray tank).

Emulsifiable concentrates (EC) or oil-in-water emulsions (EW) may be prepared by dissolving a liposome formulation in an organic solvent (optionally containing one or more wetting agents, one or more emulsifying agents or a mixture of said agents). Suitable organic solvents for use in ECs include aromatic hydrocarbons (such as alkylbenzenes or alkylnaphthalenes, exemplified by SOLVESSO® 100, SOLVESSO® 150 and SOLVESSO® 200; SOLVESSO®), ketones (such as cyclohexanone or methylcyclohexanone) and alcohols (such as benzyl alcohol, furfuryl alcohol or butanol), N-alkylpyrrolidones (such as N-methylpyrrolidone or N-octylpyrrolidone), dimethyl amides of fatty acids (such as C₈-C₁₀ fatty acid dimethylamide) and chlorinated hydrocarbons. An EC product may spontaneously emulsify on addition to water, to produce an emulsion with sufficient stability to allow spray application through appropriate equipment. Preparation of an EW involves obtaining a liposome formulation either as a liquid (if it is not a liquid at ambient temperature, it may be melted at a reasonable temperature, typically below 70° C.) or in solution (by dissolving it in an appropriate solvent) and then emulsifying the resultant liquid or solution into water containing one or more SFAs, under high shear, to produce an emulsion. Suitable solvents for use in EWs include vegetable oils, chlorinated hydrocarbons (such as chlorobenzenes), aromatic solvents (such as alkylbenzenes or alkylnaphthalenes) and other appropriate organic solvents which have a low solubility in water.

Microemulsions (ME) may be prepared by mixing water with a blend of one or more solvents with one or more SFAs, to produce spontaneously a thermodynamically stable isotropic liquid formulation. A liposome formulation is present initially in either the water or the solvent/SFA blend. Suitable solvents for use in MEs include those hereinbefore described for use in ECs or in EWs. An ME may be either an oil-in-water or a water-in-oil system (which system is present may be determined by conductivity measurements) and may be suitable for mixing water-soluble and oil-soluble pesticides in the same formulation. An ME is suitable for dilution into water, either remaining as a microemulsion or forming a conventional oil-in-water emulsion.

Suspension concentrates (SC) may comprise aqueous or non-aqueous suspensions of finely divided insoluble solid particles of a liposome formulation. SCs may be prepared by ball or bead milling the solid liposome formulation in a suitable medium, optionally with one or more dispersing agents, to produce a fine particle suspension of the compound. One or more wetting agents may be included in the composition and a suspending agent may be included to reduce the rate at which the particles settle. Alternatively, a liposome formulation may be dry milled and added to water, containing agents hereinbefore described, to produce the desired end product.

Aerosol formulations comprise a liposome formulation and a suitable propellant (for example n-butane). A liposome formulation may also be dissolved or dispersed in a suitable medium (for example water or a water miscible liquid, such as n-propanol) to provide compositions for use in non-pressurized, hand-actuated spray pumps.

A liposome formulation may be mixed in the dry state with a pyrotechnic mixture to form a composition suitable for generating, in an enclosed space, a smoke containing the compound.

Capsule suspensions (CS) may be prepared in a manner similar to the preparation of EW formulations but with an additional polymerization stage such that an aqueous dispersion of oil droplets is obtained, in which each oil droplet is encapsulated by a polymeric shell and contains a liposome formulation and, optionally, a carrier or diluent therefor. The polymeric shell may be produced by either an interfacial polycondensation reaction or by a coacervation procedure. The compositions may provide for controlled release of the liposome formulation and they may be used for seed treatment. A liposome formulation may also be formulated in a biodegradable polymeric matrix to provide a slow, controlled release of the compound.

A liposome formulation may also be formulated for use as a seed treatment, for example as a powder composition, including a powder for dry seed treatment (DS), a water soluble powder (SS) or a water dispersible powder for slurry treatment (WS), or as a liquid composition, including a flowable concentrate (FS), a solution (LS) or a capsule suspension (CS). The preparations of DS, SS, WS, FS and LS compositions are very similar to those of, respectively, DP, SP, WP, SC and DC compositions described above. Compositions for treating seed may include an agent for assisting the adhesion of the composition to the seed (for example a mineral oil or a film-forming barrier). In a seed treatment a liposomal composition can be applied in an amount of about 0.0001, 0.001, 0.01, 0.1, 1.0, 5, 10, 100, 1,000, 5,000, 10,000 g per 100 kg of seeds. For example from about 0.001 g to about 10 kg per 100 kg of seeds.

### Attractants

One or more attractants for a pest or nematode can be associated with a liposome of the invention. The density of nematodes or pests in an area in the presence of an attractant is greater than the density of nematodes or pests in corresponding area without the attractant.

Attractants for pests and nematodes, include, for example, organic acids such as formic acid, acetic acid, propionic acid, butyric acid, malic acid, oxalic acid, tartaric acid, lactic acid, avenic acid, mutagenic acid, and carbonic acid. Attractants can also be phenols such as p-hydroxybenzoic acid, p-coumaric acid, syringic acid, vanillic acid, and ferulic acid. Other attractants include, e.g., plant root tip exudates, plant boarder cell exudates, nematode sex pheromones, b-myrcene, siderophores, inorganic salts (e.g., Cl⁻, Na+, C₂H₃O₂, Mg²⁺, NH⁴⁺, SO₄(²⁻). (NH₄)₂SO₄ and MgSO₄, MgCl₂), cyclic AMP and AMP. See e.g., Riddle & Bird, Parasitology. 1985 Aug;91 (Pt 1):185-95. Attactants also include L- or D-isomers of amino acids (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine). Mollusk attractants include, for example, peptide pheromones. See, Fan et al., Brain Res. Mol. Brain Res. 48:167 (1997).

Insect attractants include, for example, coleopteran attractants (e.g.

brevicomin, dominicalure, frontalin, grandlure, ipsdienol, ipsenol, japonilure, lineatin, megatomoic acid, α-multistriatin, oryctalure,sulcatol,trunc-call), dipteran attractants (e.g., ceralure, cue-lure, latilure, medlure, moguchun, muscalure, trimedlure), homopteran attractants (e.g., rescalure), lepidopteran attractants (e.g., disparlure, codlelure, gossyplure, hexalure, litlure, looplure, orfralure, ostramone), eugenol, methyl eugenol, or siglure.

One or more attractants can be incorporated into the lipid bilayer of liposomes or can be bound to the surface of liposome.

One or more attractants can be located within lipid bilayers of liposomes by any method known in the art, e.g., osmotically. The one or more attractants can be substantially uniformly distributed within the lipid bilayers of the liposomes. In one embodiment of the invention a liposome with one or more pesticides or nematicides within the aqueous core are formed by any method known in the art (e.g., sonication, extrusion through membranes (e.g., polycarbonate filters), or French press extrusion). The liposomes can then be added to a concentrator along with one or more lipophilic attractants. The concentrator is sealed and the pressure is increased to enhance osmosis across the outer liposome layer. The pressure can be increased to about 1, 2, 3, 4, 5, 10, 15, 20, 25, 30, 35, 40, 45 atm or more (or any range between about 1 and 45 atm). Alternatively, the pressure can be increased to about 45, 40, 35, 30, 25, 20, 15, 10, 5, 4, 3, 2, 1 or less atm (or any range between about 45 and 1 atm). Pressure can be applied for 1, 5, 10, 60, 120, 300, 400, 500 minutes or more (or any range between about 1 and 500 minutes). Heat can optionally be applied. The heat should be maintained lower than the lipid bilayer phase transition temperature. For example, the temperature can be about 15, 20, 25, 30, 40, 50, 60 ° C. or more (or any range between about 15 and 60 ° C.). Alternatively, the temperature can be about 60, 50, 40, 30, 25, 20, 15 ° C. or less (or any range between about 60 and 15 ° C.).

A concentrator can be any type of vessel that can hold liposome formulations and that can be subjected to pressure and optionally, heat. The concentrator can have an inlet for a liposome sample and an optional outlet (such as a valve or a port) and elements to apply pressure, heat, or a combination thereof to a liposome sample within the vessel. The concentrator can have a valve, lid, or locking mechanism to seal the vessel so that pressure can be applied to the liposome sample. Optionally, the concentrator is located in line with equipment (e.g., an extruder) that produces liposomes with one or more pesticides or nematicides in the aqueous core. The extruder (or other liposome producing equipment) can be attached to the concentrator so that the output of the extruder (a liposome formulation) goes to the inlet of the concentrator. After treatment in the concentrator the liposomes will have one or more pesticides or nematicides in the aqueous core and one or more attractants located within the lipid bilayers.

In another embodiment of the invention one or more attractants are coupled to the surface of liposomes by any method known in the art. For example, attractants can be attached to the surface of a liposome by methods involving the use of an organic solvent, methods involving the use of mechanical means (e.g., French press), methods involving the use of detergents, or methods of direct incorporation of the attractant onto preformed liposomes.

In one embodiment of the invention, an attractant coupled to the surface of a liposome (optionally through the use of a ligand such as a protein ligand) can be anchored to the surface of the liposome such that the ligand or attractant is anchored in the core of the liposome, anchored within the lipid bilayer, or anchored to the surface of the liposome such that the all of or at least part of the attractant is on the exterior of the liposome.

Proteins or polypeptides can be conjugated to liposomes using methods based on nucleophilic reactivity of free amino groups of the proteins. A two stage coupling procedure involving carboxyacyl derivatives of phosphatidylethanolamine can be used. First, a lipidic free carboxylic group is activated with water soluble carbodiimide at pH=pKa-1, where pKa is the ionization constant of the given carboxylic group. In the second step, protein solutions are added with a simultaneous change to pH 8. Alternatively, thiol reactive phospholipid derivatives can be conjugated to liposomes with the thiol groups of proteins. N-PDP-EP and N-MPB-PE are two examples of the thiol reactive phospholipids. Many other methods are known in the art for attachment of different types of molecules to liposome surfaces. For example, “click chemistry” can be used to attach various ligands to the surface of preformed liposomes. This chemoselective reaction involves a Cu(I)-catalyzed azide-alkyne cycloaddition, which can be performed under mild experimental conditions in aqueous media. See Frisch et al., Methods in Molecular Biology Volume 605, 2010, pp 267-277.

In one embodiment of the invention an attractant is coupled to the surface of a liposome through a spacer such as a polymer or a hydrophilic polymer (e.g., polyethyleneglycol (PEG)). The spacer can be linked to a liposome by, e.g., physically adsorbing the spacer onto the surface of the liposome, by incorporating the spacer-lipid conjugate during liposome preparation, or by covalently attaching reactive groups onto the surface of preformed liposomes.

The attractant can be covalently bound to the free distal end of a spacer (e.g., a hydrophilic polymer chain), which is attached at its proximal end to the liposome, after the lipid-spacer is inserted into the liposome. There are a wide variety of techniques for attaching a polymer (e.g. PEG) to a lipid and activating the free, unattached end of the polymer for reaction with the attractant. See e.g., Allen et al., Biochemicia et Biophysica Acta, 1237:99-108 (1995); Zalipsky, Bioconjugate Chem., 4(4):296-299 (1993); Zalipsky et al. FEBS Lett., 353:71-74 (1994); Zalipsky et al., Bioconjugate Chemistry, 6(6):705-708 (1995); Zalipsky in STEALTH LIPOSOMES (Lasic & Martin, Eds.) Chapter 9, CRC Press, Boca Raton, Fla. (1995)). Where the linker is PEG the PEG can be about 100, 200, 500, 1,000, 2,000, 3,000, 4,000, 5,000 kDa or more.

### Methods of Use of Liposomes Formulations of the Invention

The liposomal formulations of the invention can reduce leaching of pesticides and nematicides into soil, can prevent migration of pesticides and nematicides through soil (due to slow release from liposomes), can prevent the binding of biological (i.e. DOBA) or chemical (i.e. Abamectin) pesticides and nematicides to organic materials, and can be formulated to bind to plant roots. The liposomal formulations of the invention therefore can help to efficiently deliver pesticides or nematicides to the site of action where pests and plants interact, thereby improving control. In addition, compositions of the invention can be formulated to control the release of pesticides and nematicides into different soil types. Compositions of the invention can also be integrated with crop rotation to control pesticides and nematicides that infect wide range of hosts. Different formulations with effective pesticide and nematicide doses can also be developed and integrated with soil textures maps to reduce pesticide and nematicide use and run off in the environment. Compositions of the invention also can be effective to control nematodes and pests in fields with varying soil textures or that need to application at different rates and different times of plant growth stages.

Formulations of the invention are effective against larvae, eggs, juveniles, and adult insects, nematodes, and other pests. Formulations of the invention can kill or paralyze insects, nematodes and pests. They can also reduce the numbers of larvae, eggs, and adult pests, insects, and nematodes on plants, plant tissues, and in or on soil or plant media.

The method of application of the compositions of the invention to soils, plant media, plants, seeds, seedlings or plant tissues is important. Compositions of the invention can be applied to soils or plant media when the plants are pre-emergent or post-emergent.

Compositions of the invention can be applied by mechanical sprayers. Sprayers convert a formulation of the invention which is mixed with a liquid carrier, such as water or fertilizer, into droplets. The droplets can be any size. Boom sprayers and air blast sprayers can also be used to apply formulations of the invention to pre-emergent or post-emergent crops. Air blast sprayers inject formulations of the invention mixed with a liquid carrier into a fast-moving air stream. Boom sprayers, aerial sprayers, ultra-low volume sprayers, drip irrigation, sprinkler irrigation, and foggers can also be used to apply formulations of the invention. Where the formulations of the invention are in a solid, powder or granule form, they can be applied with granule or dust application equipment. Liposomal formulations of the invention can also be applied to soil, plant media, plants, plant tissues or seeds as a fumigant.

In one embodiment of the invention freeze-dried or lyophilized liposomes containing one or more pesticides (e.g., nematicides) are applied directly to non-emergent crops, emergent crops, seeds, soil, plant medium, seeds or plant tissues. In another embodiment of the invention freeze-dried or lyophilized liposomes are reconstituted or rehydrated with water, another liquid (e.g., fertilizer, pesticide, herbicide, nematicide), or any other suitable liquid or gel and then applied directly to non-emergent crops, emergent crops, seeds, soil, plant medium, seeds or plant tissues. The liquid can be fertilizer or can contain fertilizer or other components.

Pesticides are usually recommended for field application as an amount of pesticide per hectare (g/ha or kg/ha) or the amount of active ingredient or acid equivalent per hectare (kg a.i./ha or g a.i./ha).

Advantageously, a much lower amount of pesticide, e.g., nematicide, is required to be applied to soil, plant media, seeds plant tissue, or plants to achieve the same results as where the pesticide is applied in a non-liposomal formulation. For example, the amount of pesticide or nematicide is applied at levels about 2, 3, 4, 5, 6, 7, 8, 9, 10, 15, 20, 30, 50, or 100-fold (or any range between about 2 and about 100-fold, for example about 2- to 10-fold; about 5- to 15-fold, about 10- to 20-fold; about 10- to 50-fold) less than the same pesticide or nematicide applied in a non-liposomal formulation, e.g., direct application of the same pesticide or nematicide. For example, oxamyl in a non-liposomal formulation has a suggested application rate for potatoes of 4.0 to 5.5 kg a.i. /ha. When oxamyl is incorporated into the liposome formulations of the invention, the application rate would fall to about 0.4 to 0.55 kg a.i./ha (10-fold less).

Liposome formulations of the invention can be applied at about 0.0001, 0.001, 0.005, 0.01, 0.1, 1, 2, 10, 100, 1,000, 2,000, 5,000 (or any range between about 0.0001 and 5,000) kg/ha. For example, about 0.0001 to about 0.01, about 0.01 to about 10, about 10 to about 1,000, about 1,000 to about 5,000 kg/ha.

Surprisingly, where pesticides or nematicides in the liposomal formulations of the invention are applied at the same concentration as non-liposomal formulations, the liposomal formulations have unexpected advantages. Firstly, liposomal formulations of the invention when applied at the same concentrations as non-liposomal formulations are more effective at controlling pests and nematodes and at reducing damage to plants such as gall formation and root necrosis. Secondly, the use of liposomal formulations of the invention applied at the same or lower concentrations as non-liposomal formulations result in longer root length of plants, enhanced stalk growth, enhanced leaf growth, and healthier plants having enhanced vigor.

Therefore, the invention includes methods of increasing root lengths, increasing stalk diameter, increasing stalk length, increasing leaf number, increasing leaf size of a plant, increasing plant vigor or a combination thereof of a nematicide or pesticide treated plant or a plant grown in nematicide- or pesticide-treated soil or plant media comprising administering one or more nematicides or pesticides to the plant or the soil or plant media, wherein the one or more nematicides or pesticides are present in an aqueous core of a liposome. The nematicides or pesticides present in the aqueous core of a liposome can be administered at a same amount or a lower amount or concentration than the recommended administration amount or concentration of the same nematicide or pesticides when administered in a non-liposomal formulation.

Another embodiment of the invention provides a method of decreasing the amount of nematicide—or pesticide-induced damage to nematicide or pesticide treated plants or a plants grown in nematicide—or herbicide—treated soil or plant media comprising administering one or more nematicides or pesticides to the plant or the soil or plant media, wherein the one or more nematicides or pesticides are present in an aqueous core of a liposome. The nematicides or pesticides present in the aqueous core of a liposome can be administered at a same amount or concentration or a lower amount or concentration than the recommended administration amount or concentration of the same nematicides or pesticides when administered in a non-liposomal formulation.

Nematicide—or pesticide—induced damage to plants can include, for example, root necrosis, gall formation, decreased yields, less developed root system (including shorter roots), tillering decrease, decrease in plant height, decrease in stalk circumference, smaller leafs, less leaves, more dead basal leaves, more fertilizers needed, more seeds needed, less productive tillers, later flowering, later grain, seed or fruit maturity, more plant verse (lodging), decreased shoot growth, decreased plant vigor, or a combination thereof.

### Treatment of Humans and Animals

Liposomal compositions of the invention can also be used to treat animals, including mice, rats, horses, cattle, sheep, pigs, dogs, cats, and primates. The compounds of the invention are also effective for use in humans. Administration of the liposomal compositions can reduce or alleviate the symptoms of an animal infected with or in contact with one or more pests or nematodes. Administration can also eliminate or reduce the number of pests or nematodes infecting or in contact with an animal.

The liposomes of the present invention can be administered by any suitable means including, but not limited to, for example parenterally, intraarticularly, subcutaneously, intramuscularly, intradermally, intravenously (including an intravenous drip), intraperitoneally (including bolus injection), intramedullary, intrathecally, intraventricularly, transdermally, subcutaneously, intranasally, orally, rectally, topically (including transdermal, aerosol, buccal and sublingual), vaginally, or intravesically.

Liposomes of the invention can be present in a pharmaceutical formulation. For example, in addition to the active ingredients, liposomal pharmaceutical compositions of the invention can contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

The concentration of liposomes in the pharmaceutical formulations can vary widely, i.e., from less than about 0.05%, usually at or at least about 2-5% to as much as 10 to 30% by weight and will be selected primarily by fluid volumes, viscosities, etc., in accordance with the particular mode of administration selected. For example, the concentration can be increased to lower the fluid load associated with treatment. Alternatively, liposomes can be diluted to low concentrations to lessen inflammation at the site of administration. The amount of liposomes administered will depend upon the particular nematicides or pesticides used, the disease state being treated and the judgment of the clinician, but will generally, in a human, will be between about 0.001 and about 50 mg per kilogram of body weight, for example, between about 0.001 and 10 mg/kg or between about 5 and about 40 mg/kg of body weight. Higher lipid doses are suitable for other animals, for example, 50-120 mg/kg.

Dosage for the liposomal compositions will depend on the administrating physician's opinion based on age, weight, and condition of the patient, and the treatment schedule. Doses of pesticides or nematicides in humans will be effective at ranges as low as from 0.015 mg/M²/dose and will still be tolerable at doses as high as 15 to 75 mg/M²/dose, depending on dose scheduling. Doses may be single doses or they may be administered repeatedly every 4 h, 6 h, or 12 h or every 1 d, 2 d, 3 d, 4 d, 5 d, 6 d, 7 d, 8 d, 9 d, 10 d or combination thereof. Scheduling may employ a cycle of treatment that is repeated every week, 2 weeks, three weeks, four weeks, five weeks or six weeks or combination thereof.

In certain embodiments, the liposomal compositions of the invention can be administered as a preventative measure. Prevention or preventing refers to a reduction of the risk of acquiring a pest or nematode infection. The compositions of the invention can be administered as a preventative measure to a subject even though symptoms of pest or nematode infection are absent or minimal.

About, as used herein, means that the value varies up or down by 5%. For example, for a value of about 100, means 95 to 105 (or any value between 95 and 105).

All patents, patent applications, and other scientific or technical writings referred to anywhere herein are incorporated by reference herein in their entirety. The invention illustratively described herein suitably can be practiced in the absence of any element or elements, limitation or limitations that are not specifically disclosed herein. Thus, for example, in each instance herein any of the terms “comprising”, “consisting essentially of”, and “consisting of” may be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by embodiments, optional features, modification and variation of the concepts herein disclosed may be resorted to by those skilled in the art, and that such modifications and variations are considered to be within the scope of this invention as defined by the description and the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

The following are provided for exemplification purposes only and are not intended to limit the scope of the invention described in broad terms above.

### EXAMPLES

### Example 1

### Egg Extraction

M. incognita eggs collected from tomato cultures by NaOC1 extraction (Hussey and Barker, 1973). Briefly, six to twelve week old infected tomato roots were cut into 1-2 segment. Root segments were shacked vigorously in 200 ml of a 0.5% Na O Cl solution for 4 min. Then, the Na O Cl solution was passed quickly through a 200-mesh (75-μm), nested over a 500-mesh sieve to collect freed eggs. The a 500-mesh sieve with eggs was quickly placed under a stream of cold water to remove residual Na O cl and rinsed several times. About 50 ml queous suspension of eggs was collected and number of eggs per unit volume will be counted. The egg suspension was allowed to sit at room temperature for 4 days. The hatching juveniles were collected and used in the subsequent experiments.

### Example 2

### Liposomes Preparations

Liposomes having an aqueous core and phospholipid bilayers were prepared using the thin-film dehydration-rehydration method obtaining, multilamilar vesicles (MLVs) and small unilamellar vesicles (SUVs) (Bangham et al., J. Mol. Biol. 13:238-252 (1965); Gosangari & Watkin, Pharm Dev Technol. 17:383-8 (2012)). Using thin-film hydration method, briefly, required amounts of lipids [1,2-Dipalmitoyl-sn-glycero-3-phosphocholine (DPPC) liposomes and 1, 2-distearoyl-snglycero- 3-phosphocholine (DSPC)] were dissolved in chloroform, and a thin film will be formed on the inner side of the round bottom flask, by evaporating the solvent under vacuum using a rotavapor. The lipid film formed was stored overnight in vacuum desiccator to eliminate traces of chloroform. The film was then hydrated at 58° C., above the Tc of DSPC, using 10 mL of phosphate-buffered saline (PBS, 20 mM Na2HPO3-NaH2PO3, 150 mM NaCl, pH 7.0) containing different concentration of Oxamyl. The hydration process of Oxamyl liposomes was done with vigorous agitation to form multilamellar vesicles (MLV). The formed liposomes were sonicated using a probe sonicator in 5 cycles of 2 min each. The MLVs were centrifuged at 10000×g for 15 minutes to purify the liposomes from the un-encapsulated Oxamyl (Mohammed et al., Int J Pharm. 285:23-342004). To form small unilamellar vesicles (SUV), the multilamellar liposomes were extruded through polycarbonate membranes of pore sizes 1.0 μm (Olson et al., Biochim Biophys Acta. 557:9-23 (1979)). The unencapsulated oxamyl was then separated using Sephadex G-50 macrospin columns and the encapsulation efficiency was calculated spectrophotometrically at 280 nm. The liposomal fractions collected from the Sephadex columns were pooled and lyophilized after addition of suitable amount of sucrose as a cryoprotectant. Addition of sugars to liposome formulation prevent vesicle fusion and have been attributed to the formation of a stable glassy state as well as direct interaction between the polar head groups of phospholipids and sugars (Crowe & Crowe,

Biochim Biophys Acta. 939:327-34 (1988)).

### Example 3

### Fluorescent Uranin-Liposomes:

We have developed a micron sized liposome that has the ability to encapsulate different concentrations of oxamyl (or other insecticide/nematicide). It is an efficiency method that suppresses the root-knot nematodes. To prove this concept we used liposomes loaded with 100 mM of the hydrophilic fluorescent reagent uranin to test oral administration of water-soluble substances to the plant parasitic nematode. Liposomes prepared as mentioned above. Uranin solution (2 mg/ml) in PBS buffer was added to thin film of liposomes during rehydration at above 50° C. Unencapsulated uranin was removed through gel chromatography. About 50 ul of uranin liposome solution was mixed with nematode suspension and incubated for 2-3 days at room temperature. The mix was then visualized by fluorescent microscope. Our data demonstrate that ingestion of liposomes loaded with fluorescent dye resulted in successful oral delivery of chemicals into the intestines of Root-knot and Spiral nematodes. Spiral nematodes fed 25 μl of liposomes containing uracin showed clear fluorescence along their esophagus digestive tracts. Root nematodes fed 25 μl of liposomes containing uracin showed clear fluorescence along several organs of their bodies.

### Example 4

### Effect of Oxamyl (Vydate) and Thiocarb (Larvin) Liposome Formulation on Root-Knot Nematodes in Vitro

Determine the efficient concentration of nematicides (Oxamyl and Thidiocarb) that kill or suppress Root-Knot Larvae.

M. incognita eggs collected from tomato cultures by NaOC1 extraction (Hussey & Barker, Plant Disease Reporter. 57:1025-1028 (1973)). Egg suspension was incubated at room temperature until larvae were hatched (4-5 days). The juveniles (J2) were counted and evaluated for activity/mobility for the duration of the study. Four different concentrations of both nematicides (Oxamyl and Thiodicarb) were used to assess their efficacy in killing the nematodes. These were untreated control, 200 ug, 1 mg, 2 mg, and 10 mg. Three replicates of the each concentration were mixed with J2 suspensions and incubated at room temperature for 2 days.

Root-Knot larvae (J2)

| Treatment | Alive | Dead |
|---|---|---|
| Control 1 | 97 | 3 |
| Control 2 | 93 | 7 |
| Control 3 | 100 | 0 |
| Larvin (Thiodicarb) 200 ug | 76 | 23 |
| Larvin (Thiodicarb) 200 ug | 86 | 14 |
| Larvin (Thiodicarb) 200 ug | 89 | 11 |
| Larvin (Thiodicarb) 1 mg | 63 | 37 |
| Larvin (Thiodicarb) 1 mg | 75 | 25 |
| Larvin (Thiodicarb) 1 mg | 82 | 18 |
| Larvin (Thiodicarb) 2 mg | 70 | 30 |
| Larvin (Thiodicarb) 2 mg | 85 | 15 |
| Larvin (Thiodicarb) 2 mg | 65 | 35 |
| Larvin (Thiodicarb) 10 mg | 70 non-mobile | 30 |
| Larvin (Thiodicarb) 10 mg | 80 non-mobile | 20 |
| Larvin (Thiodicarb) 10 mg | 70 non-mobile | 30 |
| Oxamyl (Vydate) 200 ug | 60 non-mobile | 40 |
| Oxamyl (Vydate) 200 ug | 58 non-mobile | 42 |
| Oxamyl (Vydate) 200 ug | 50 non-mobile | 50 |
| Oxamyl (Vydate) 1 mg | 0 | 100 |
| Oxamyl (Vydate) 1 mg | 0 | 100 |
| Oxamyl (Vydate) 1 mg | 0 | 100 |
| Oxamyl (Vydate) 2 mg | 0 | 100 |
| Oxamyl (Vydate) 2 mg | 0 | 100 |
| Oxamyl (Vydate) 2 mg | 0 | 100 |
| Oxamyl (Vydate) 10 mg | 0 | 100 |
| Oxamyl (Vydate) 10 mg | 0 | 100 |
| Oxamyl (Vydate) 10 mg | 0 | 100 |

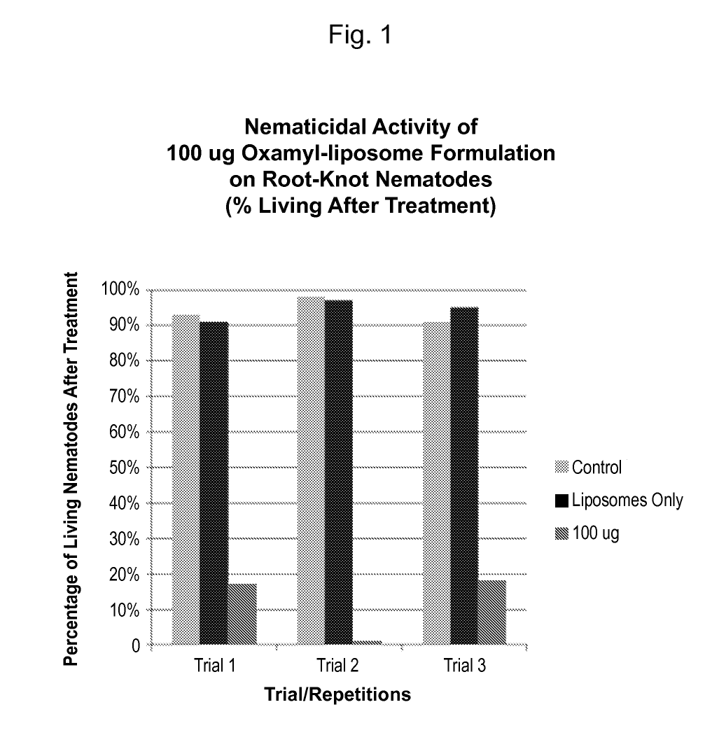

Based on the data mentioned above, we eliminated Thiodicarb (Larvin) because it required higher concentration to kill Root-Knot (J2). We studied the efficiency of Oxamyl; 200 ug/ml and 100 ug/ml in suppression of J2. We found that both concentrations lead to 100% mortality of J2 larvae. We used 100 ug of Oxamyl in subsequent studies with liposomes. Oxamyl-liposome formulation was created and it demonstrated its efficiency to suppress Root-knot nematodes as follows.

| | |
|---|---|
| | Nematicidal activity of 100 ug/ml Oxamyl- |
| Treatment | liposome formulation on root-knot nematodes |

| Control 1 | 7% | dead |
|---|---|---|
| Control 2 | 2% | dead |
| Control 3 | 9% | dead |
| Liposomes only 1 | 9% | dead |
| Liposomes only 2 | 3% | dead |
| Liposomes only 3 | 5% | dead |
| 100 ug | 83 dead 17 non mobile | |
| 100 ug | 100 | dead |

| | |
|---|---|
| 100 ug | 82 dead 18 non mobile |

Where no oxamyl was added, the larvae were free and active. Where oxamyl was added the larvae were dead or paralyzed. See also, FIG. 1. FIG. 1 shows the nematicide activity of 100 μg of oxamyl-liposome formulations on root-knot nematodes.

### Dosage

The lethal effect of nematicides is determined by two components. The first is concentration (C) of the nematicide in soil solution, usually expressed as 5 parts per million (PPM). The second is the length of time (T) the nematode is exposed, expressed in minutes, hours or days. The level of nematode control is then related to dosage, the amount of pesticide placed in the environment of the nematode for a known length of exposure time (concentration×time). Total exposure is the sum of CT products.

For most organisms, nematodes included, there is a nematicide concentration level, below which kill is not obtained regardless of the length of exposure. If exposure to 10 ppm for 20 days (200 CT) is the minimum dosage required to kill a nematode, then exposure to 4 ppm for 50 days (200 CT) will be totally ineffective even though the nematode has received the same cumulative dosage. In this example, a minimum concentration of 10 ppm was required to effectively contribute to the lethal or disorientating activity of the nematicide. For most nematodes, long exposures to low concentrations of fumigant nematicides above the minimum concentration appear to be more effective than short exposures to higher concentration. All nematode species are not equally susceptible to a given nematicide nor are all life stages of a given species equally sensitive given the same exposure time. For example, after a 24 hour exposure to the fumigant nematicide EDB, only 75% of a population of free living nematodes in soil was killed while the citrus nematode did not survive a 0.5 hour exposure to EDB at the same concentration. In dry soils, many nematodes which can survive in a dehydrated state can tolerate 10 times the lethal dose of methyl bromide compared to active forms in moist soil. In practice, fumigant nematicides are commonly injected through a series of uniformly spaced shanks into soil. As the liquid volatilizes, gases begin moving in mass flow, diffusing radially outward in all directions from the point of injection. Since diffusion is greater in air above the soil surface, upward mass flow and diffusion is usually greater than downward movement, and much of the gas may escape the soil and enter the atmosphere. As the nematicide front moves through soil, gaseous molecules are adsorbed to particle surfaces, redissolve into soil solution, and fill empty air spaces between soil particles. Maximum nematicide concentration decreases as do the sums of CT products with distance from the point of injection. Eventually, with time and distance, concentrations fall below an immediate killing level. The number of nematodes killed by fumigant treatment within these areas depends on the number of CT units which develop within the nematicide treated zone.

The relationship between nematicide application rate and nematode control is therefore not only a measure of pesticide toxicity but chemical dispersion as well. If dispersal is good, increases in chemical application rates will result in higher CT values and provide control to a greater soil volume. If dispersal is poor, increases in application rates will not provide control to a larger soil volume. Unlike fumigant nematicides where water may effectively block efficient dispersion in soil, nonfumigant nematicides must be carried by rainfall or irrigation water into soil to be effective. Nematicide concentration and its persistence above a certain effective concentration is also important for nematode control with nonfumigant nematicides. The apparent failure to control nematodes with nonfumigant nematicides in many instances, is very likely the result of excessive rainfall or irrigation and poor chemical retention within the primary rooting zone of the crop. Unless otherwise explained, all technical and scientific terms used herein have the same meaning as commonly understood by one of ordinary skill in the art to which a disclosed disclosure belongs.

### Example 5

### Pre-Emergent Soil Treatment

The use of the liposomal formulations for the pre-emergent treatment of soil was tested. Peat chips were placed in a container with 3 rows of 5 chips each. The chips were hydrated with tap water and kept at room temperature for two days. The experimental and control treatments were administered to the wood chips. The chips were allowed to rest for one or two days. Two to three tomato seeds were planted on each chip. The plant stalk and leaf growth was monitored during plant emergence.

The normal control condition comprised no delivery of any type of Avid 0.15

(Syngenta) to the chips. For the experimental conditions one of three dosage levels of liposomal Avid 0.15 were applied to the chips:

(1) 56 μl, which is the commercially recommended dose level adjusted for the given area of the test chips;

(2) 1 μl

(3) 0.5 μl.

The doses were applied to the center of the chip using a Gilson Pipette Man.

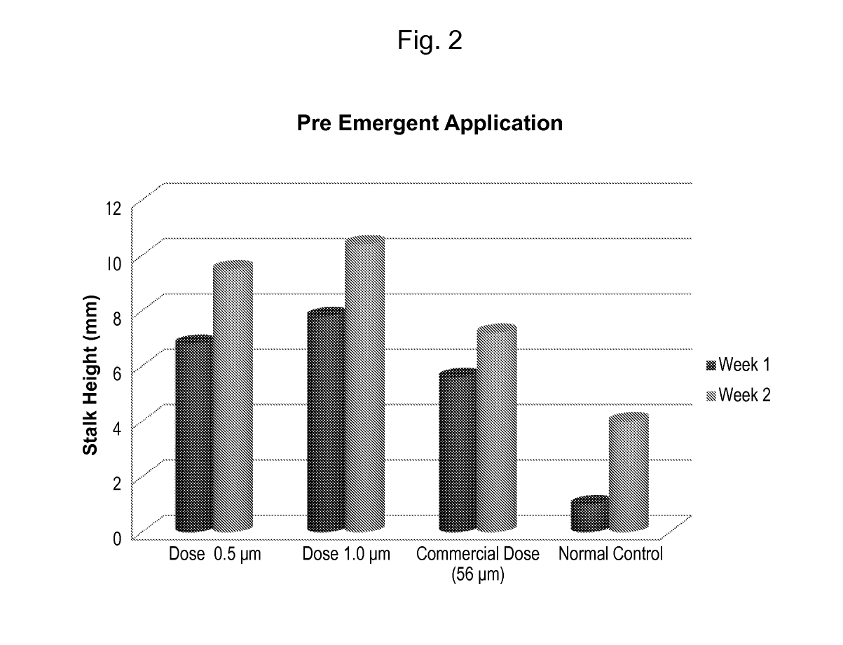

The results are shown in FIG. 2. Pre-emergent treatment of the chips with the liposomal formulations enhanced emerging plant growth compared to the untreated normal growth conditions. Lower doses (1 μl and 0.5 μl) enhanced stalk and leaf growth and lead to healthier plants as compared to the higher dose (56 μl). The plants administered the higher dose (56 μl) performed better than the normal control, but had inhibited leaf growth as compared to the normal control plants. At two weeks from planting the normal controls had large multi-leaf growth and strong stalks. The high dose (56 μl) plants had thin stalks and very small leaves. The low dose plants (1μl) had multiple large leaves. The very low dose plants (0.5 μl) had medium to large multi-leaf growth.

### Example 6

### Abamectin Liposomal Formulations

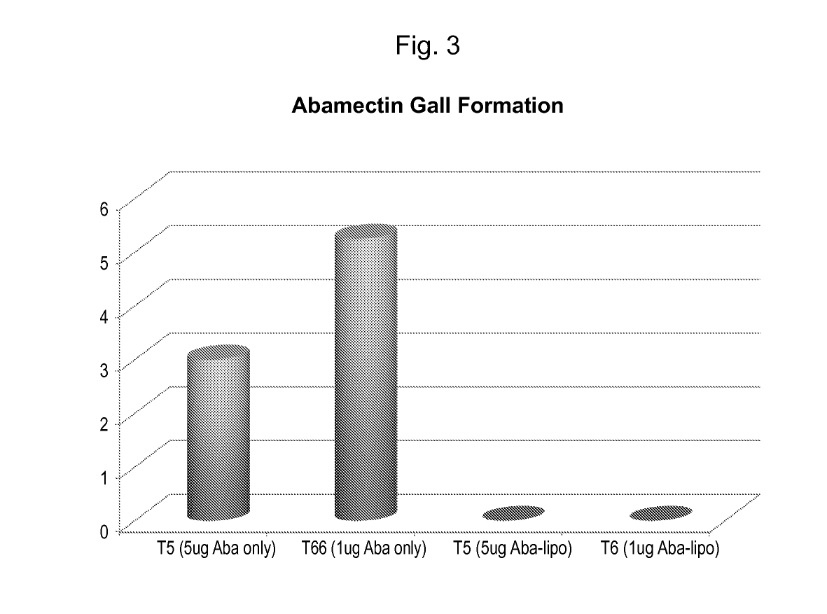

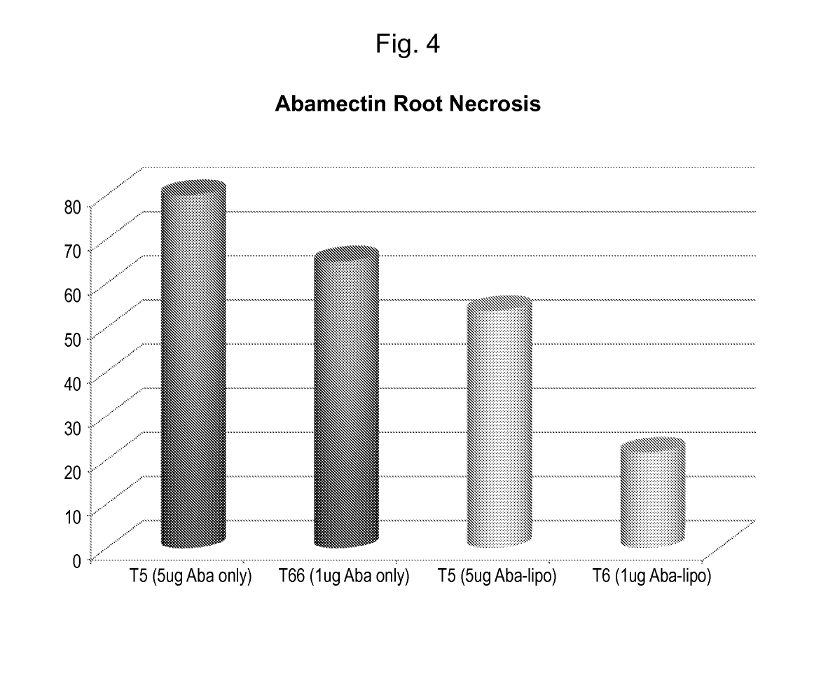

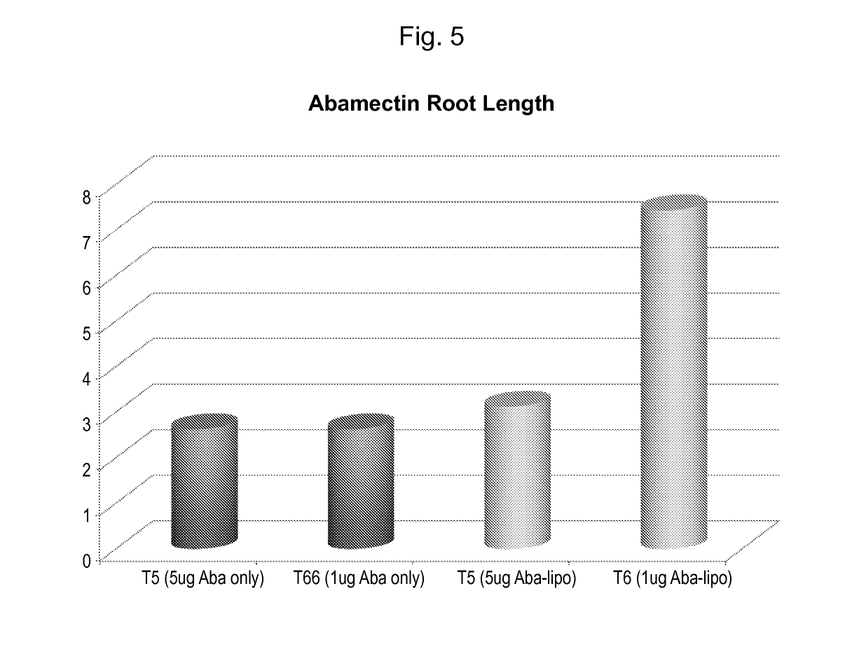

Abamectin at 5μg or 1μg was directly applied to soil prior to planting. Alternatively abamectin was loaded into liposomes at either 5μg or 1 μg and applied to soil prior to planting. Gall formation was detected. The results are shown in FIG. 3. The non-liposomal abamectin 1μg application resulted in the most gall formulation followed by the non-liposomal abamectin 5μg application. The liposomal abamectin 5μg or 1μg applications had almost non-detectable levels of gall formation. Additionally, the non-liposomal abamectin 5μg application resulted in the most root necrosis followed by the non-liposomal abamectin 1μg application. The liposomal abamectin 5μg or 1μg applications resulted in less root necrosis. See FIG. 4. Additionally, the liposomal abamectin 5μg or 1μg applications resulted in longer root length than for the non-liposomal abamectin 1 μg or 5 μg applications. See FIG. 5. Therefore, liposomal abamectin formulations enhance root length as compared to non-liposomal abamectin formulations.

### Example 7

Four replicates for each condition of Rutgers tomato plants were inoculated with 1,000 J2 and 2000 eggs of Root Knot nematodes. At 2-3 weeks old, the plants were treated with at 3 dose levels (5 μg, 1, μg, and 500 ng) of either abamectin or liposomal encapsulated abamectin. Plants were harvested 5-6 weeks after treatment.

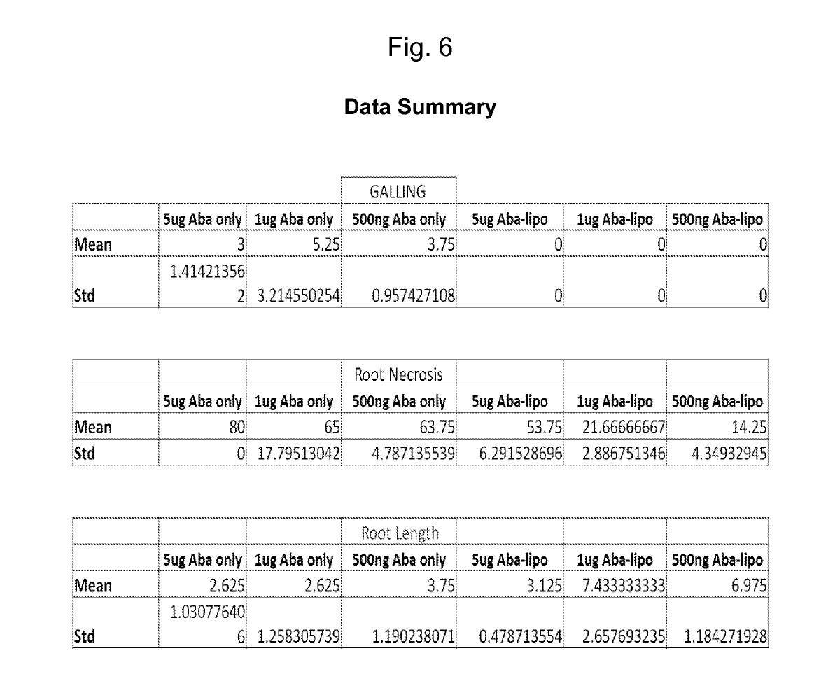

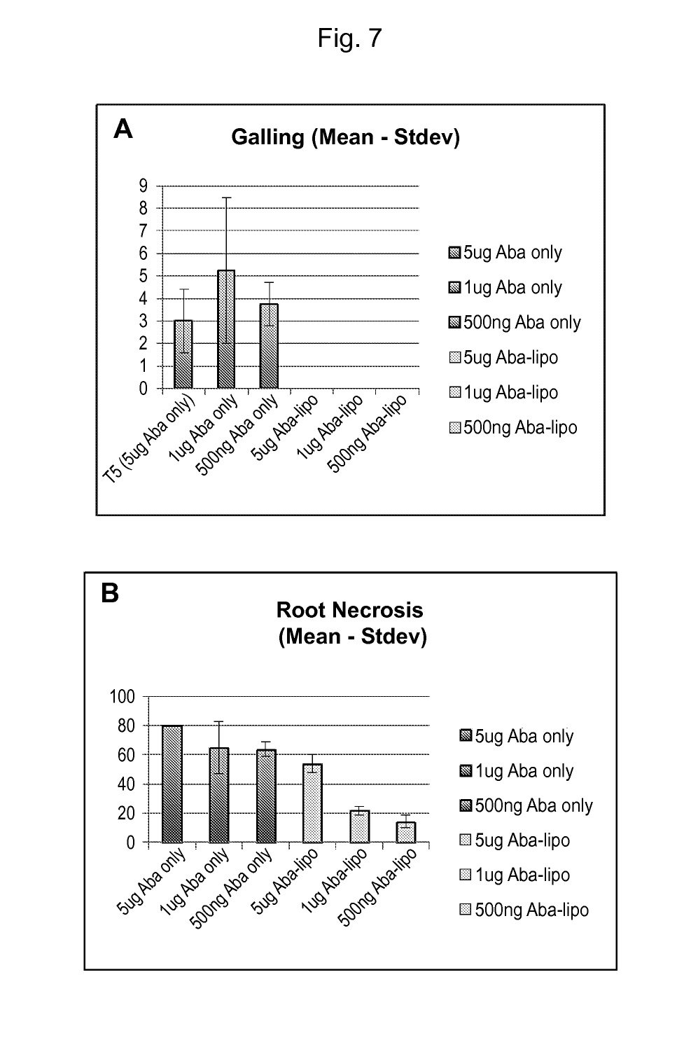

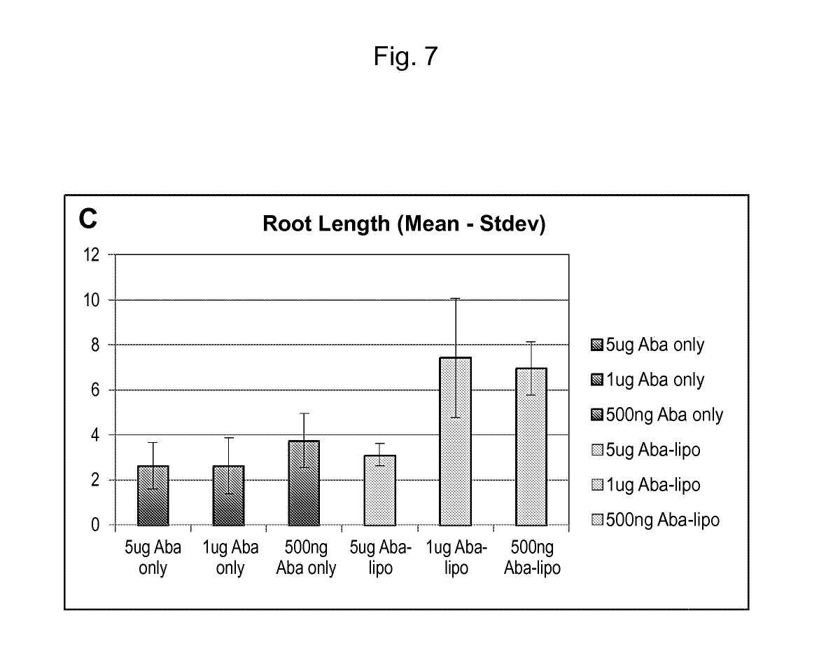

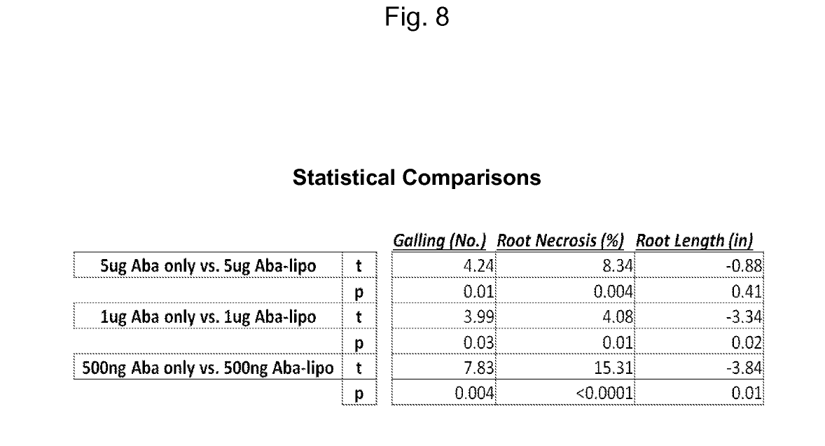

The summary of the results on galling, root necrosis, and root length is shown in FIG. 6. FIG. 7A-C shows graphs of the number of galls, percentage of root necrosis, and root length (in inches). FIG. 8 shows the statistical comparisons between the treatment types. The reduction in root necrosis and increased root length would provide enhanced crop yield.

It is estimated that the addition of an attractant to the liposomal formulation would result in a further reduction of in root necrosis by 50% and substantially increase root length by about 25%. This would provide an enhanced crop yield.

### Example 8

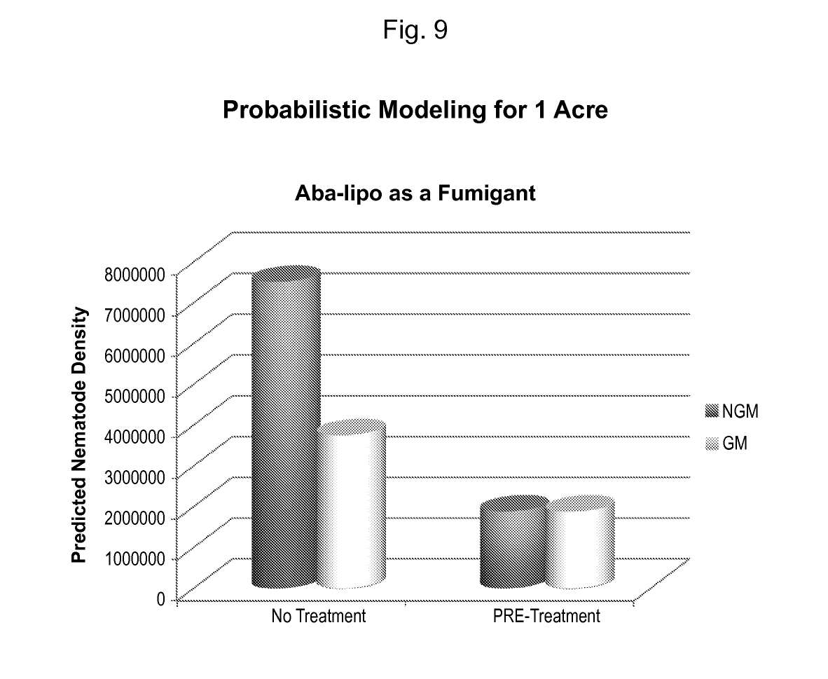

Abamectin encapsulated liposomes were used in a field test of genetically modified soybeans and non-genetically modified soybeans. The control and treatment microplots were 10 feet by 20 feet. A 100 μg concentration of abamectin encapsulated liposomes were sprayed as a fumigant evenly onto treatment plots two days prior to planting. A Bayesian approach was taken and a hierarchical model was used to predict the nematode reduction per acre. The results are shown in FIG. 9.

Abamectin encapsulated liposomes substantially reduce nematode populations. About 23 mg of abamectin encapsulated liposomes can be used to decrease the nematode load by 50 to 75% for an acre of soybeans. This is a substantial reduction in the amount of abamectin needed to treat nematodes when it is not encapsulated by liposomes (about 2982 mg/acre).

It is estimated that the addition of an attractant to the liposomal formulation would result in further reduction of nematode population by about 50%.

We claim:

1. A liposome formulation comprising one or more pesticides or nematicides loaded in the aqueous core of liposomes, wherein one or more attractants for a pest or nematode are present within the lipid bilayer or bound to the surface of the liposome.

2. The liposome formulation of claim 1, wherein the liposomes are lyophilized or frozen.

3. The liposome formulation of claim 1, wherein the one or more nematicides are 2-methyl-2-(methylthio)propionaldehyde O-methylcarbamoyloxime, 2, 3-D i hydro-2,2-d im ethyl-7-benzofuranyl methylcarbamate, 2-methyl-2-(methylsulfonyl)propanal-O-(methylaminocarbonyl oximel, O,O-diethyl O-[p-(methylsulfinyl)phenyl] ester, Ethyl 4-methylthio-m-tolyl isopropylphosphoramidate, O-ethyl S,S-dipropyl phosphorodithioate, Methyl N′N′-dimethyl-N-[(methylcarbamoyl)oxy]-1-thiooxam im idate, S-[[(1,1-dimethylethyl) thio] methyl]O,O-diethyl phosphorodithioate, thionazin, isazofos, ebufos, cleothocarb or combinations thereof.

4. The liposome formulation of claim 2, wherein the lyophilized or frozen liposome is loaded with about 1, 5, 10, 50, 100, 200, or 500 pg/ml of the one or more pesticides or nematicides.

5. The composition of claim 1, wherein the liposome formulation is a dustable powder (DP), soluble powder (SP), water soluble granules (SG), water dispersible granules (WG), wettable powders (WP), granules (GR) (slow or fast release), soluble concentrates (SL), oil miscible liquids (OL), ultra-low volume liquids (UL), emulsifiable concentrates (EC), dispersible concentrates (DC), emulsions (both oil in water (EW) and water in oil (EO)), micro-emulsions (ME), suspension concentrates (SC), aerosols, fogging/smoke formulations, capsule suspensions (CS), powder for dry seed treatment (DS), a water soluble powder (SS), a water dispersible powder for slurry treatment (WS), a flowable concentrate (FS), a liquid solution (LS), a capsule suspension (CS), or combinations thereof.

6. The liposomal formulation of claim 1, further comprising a fertilizer.

7. A method for reducing the number of pests or nematodes on or in plant media, soil, plants, plant tissues, or seeds, comprising administering to the plant media, soil, plants, plant tissues, or seeds an effective amount of the liposome formulation of claim 1.

8. The method of claim 7, wherein the lyophilized liposomes are rehydrated before they are administered to the plant media, soil, plants, plant tissues, or seeds.

9. (canceled)

10. The method of claim 7, comprising administering about 5-fold, 10-fold, or 50-fold less pesticide or nematicide via the liposome formulation than is recommended for conventional, non-liposomal application of the same pesticides or nematicide.

11. The method of claim 7, wherein the plants or plants grown in the soil or plant media have increased root lengths, increased stalk diameter, increased stalk length, increased leaf number, increased leaf size, increased yield, or increased vigor as compared to plants or soil or plant media treated with non-liposomal formulations of the same one or more pesticides or nematicides of the administered liposome formulation.

12. The method of claim 7, wherein the liposome formulation is administered in an amount from about 5 g/ha to about 2000 g/ha.

13. The method of claim 7, wherein the nematodes are root-knot nematodes.

14. The method of claim 7 wherein the liposomal composition is applied to seeds in an amount from about 0.001 g to about 10 kg per 100 kg of seeds.

15. A method of increasing root lengths, increasing stalk diameter, increasing stalk length, increasing leaf number, increasing leaf size of a plant, increasing yield, increasing plant vigor, or a combination thereof comprising administering a composition of claim 1 to the plant or to soil or plant media in which the plant is growing.

16. A method of increasing root lengths, increasing stalk diameter, increasing stalk length, increasing leaf number, increasing leaf size of a plant, increasing yield, increasing plant vigor or a combination thereof of a pesticide-treated or nematicide-treated plant or a plant grown in pesticide-treated or nematicide-treated soil or plant media comprising administering one or more pesticides or nematicides to the plant or the soil or plant media, wherein the one or more pesticides or nematicides are present in an aqueous core of a liposome that has one or more attractants for a pest or nematode present within the lipid bilayer or bound to the surface of the liposome.

17. The method of claim 1, wherein the pesticides or nematicides present in the aqueous core of a liposome are administered at a same amount or concentration or a lower amount or concentration than the recommended administration amount or concentration of the pesticide or nematicide when administered in a non-liposomal formulation.

18. A method of decreasing the amount of pesticide-induced or nematicide-induced damage to pesticide or nematicide treated plants or plants grown in pesticide-treated or nematicide-treated soil or plant media comprising administering one or more pesticides or nematicides to the plant or the soil or plant media, wherein the one or more pesticides or nematicides are present in an aqueous core of a liposome that has one or more attractants for a pest or nematode present within the lipid bilayer or bound to the surface of the liposome.

19. The method of claim 18, wherein the pesticides or nematicides present in the aqueous core of a liposome are administered at a same amount or concentration or a lower amount or concentration than the recommended administration amount or concentration of the pesticide or nematicide when administered in a non-liposomal formulation.

20. A method for reducing the number of nematodes on or in an animal, comprising administering to the animal an effective amount of the liposome formulation of claim 1.

21. (canceled)
